(19) United States
(12) Patent Application Publication
(10) Pub. No.: US 2025/0000851 A1
(43) Pub. Date: Jan. 2, 2025

(54) ORGANIC COMPOUND (OXIME) TO COMBAT CHEMICALS CAUSING POISONING OF A NERVOUS SYSTEM OF A HUMAN BY INHIBITING AN ENZYME IN THE NERVOUS SYSTEM LEADING TO AN ACCUMULATION OF ACETYLCHOLINE, A NEUROTRANSMITTER, AND OVERSTIMULATION OF THE NERVOUS SYSTEM, LEADING TO SYMPTOMS SUCH AS MUSCLE TWITCHING, CONULSIONS, AND RESPIRATORY FAILURE

(71) Applicant: Prepaire Labs Limited, Dublin (IE)

(72) Inventors: Carl Freer, Bloomfield Hills, MI (US); Vicente Jorge Ribas, Barcelona (ES)

(21) Appl. No.: 18/144,191

(22) Filed: May 6, 2023

(51) Int. Cl. A61K 31/47

(52) U.S. Cl. CPC A61K 31/47

(57) ABSTRACT

The present invention is an organic compound (oxime) used to combat chemicals causing poisoning of a nervous system of a human by inhibiting an enzyme in the nervous system leading to an accumulation of acetylcholine, a neurotransmitter, and overstimulation of the nervous system, leading to symptoms such as muscle twitching, convulsions, and respiratory failure, said organic compound comprising (NE)-N-[1-(benzenesulfonyl)quinolin-2-ylidene]hydroxylamine (O═S(═O)(c1ccccc1)n1/c(═N/O)ccc2ccccc21).

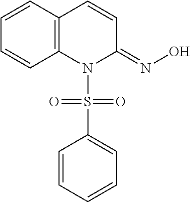 [SMILES: O=S(=O)(c1ccccc1)n1/c(=N/O)ccc2ccccc21]

### FIELD OF THE INVENTION

The present invention relates to a compound or drug for treating or preventing an organophosphate poisoning resulting from exposure to organophosphate compounds, which are commonly found in insecticides, herbicides, and chemical nerve agents.

### BRIEF DESCRIPTION OF THE INVENTION

Organophosphate poisoning is a serious medical condition resulting from

exposure to organophosphate compounds, which are commonly found in insecticides, herbicides, and chemical nerve agents such as VX and sarin. These compounds inhibit the enzyme acetylcholinesterase (AChE), causing an accumulation of acetylcholine in synapses and neuromuscular junctions. This overstimulation of cholinergic receptors leads to a range of symptoms and can be fatal if left untreated.

The symptoms of organophosphate poisoning can be classified into three categories: muscarinic, nicotinic, and central nervous system (CNS) effects. Muscarinic effects include increased salivation, lacrimation, urination, defecation, gastrointestinal motility, and bronchoconstriction. Nicotinic effects involve muscle twitching, weakness, and eventually paralysis. CNS effects manifest as seizures, confusion, ataxia, respiratory depression, and coma. In severe cases, respiratory failure and death may occur.

Organophosphate poisoning nerve agents (i.e., Sarin, Soman, or Tabun) are the most dangerous agents known, triggering seizure activity in the brain and leading to irreversible seizure-related brain damage.

Organophosphate poisoning induces rapid death through different mechanisms, including respiratory and cardiovascular significant dysfunctions. Organophosphate poisoning nerve agents primarily act by inhibiting the enzyme acetylcholinesterase (AChE), causing an acute cholinergic crisis. Accordingly, medical countermeasures (MCMs) aim to minimize the cholinergic crisis upon deactivating AChE at the neuro synaptic and neuromuscular junctions or to remove the neurotoxic agent by some scavenging process.

Alluding to the above, organophosphates are chemicals that can cause poisoning by inhibiting an enzyme called acetylcholinesterase (AChE) in the nervous system. This leads to an accumulation of acetylcholine, a neurotransmitter, and overstimulation of the nervous system, leading to symptoms such as muscle twitching, convulsions, and respiratory failure.

Despite the efforts to develop new oximes, the standard of care still relies upon a procedure dating from 1960 consisting of a combination of anti-cholinergic drugs (e.g. atropine), pralidoxime (2-PAM), obidoxime and benzodiazepines (e.g. diazepam).

One of such prior art references, such as U.S. Pat. No. 10,172,870B2 to Doodipala Samba Reddy, teaches compositions and methods for treating and/or reversing organophosphate intoxication, manifested by both cholinergic and non-cholinergic crisis, in a mammal resulting from exposure to organophosphate compounds. The neurosteroidal compounds of this invention are those having the general structural formula of pregnane, androstane, 19-norandrostanes, and norpregnane with further moieties as defined herein. These compounds include, but are not limited to, ganaxolone, pregnanolone, and androstanediol and their analogs, salts and prodrugs. The present invention further relates to combining a therapeutically effective amount of a neurosteroidal compound with a standard organophosphate antidote (e.g. atropine, pralidoxime). The data suggests that neurosteroids are effective or more effective than benzodiazepines, whether given earlier or later than 40-min (up to several hours) after organophosphate compound exposure. Neurosteroids are effective to attenuate long-term neuropsychiatric deficits caused by organophosphate exposure.

To the extent as being effective, there is always a need for new and improved compounds or drugs for treating or preventing an organophosphate poisoning resulting from exposure to organophosphate compounds, which are commonly found in insecticides, herbicides, and chemical nerve agents. The present invention aims to solve this problem by simple and convenient means.

### SUMMARY OF THE INVENTION

An organic compound (oxime) used to combat chemicals causing poisoning of a nervous system of a human by inhibiting an enzyme in the nervous system leading to an accumulation of acetylcholine, a neurotransmitter, and overstimulation of the nervous system, leading to symptoms such as muscle twitching, convulsions, and respiratory failure, said organic compound comprising (NE)-N-[1-(benzenesulfonyl)quinolin-2-ylidene]hydroxylamine (O═S(═O)(c1ccccc1)n1/c(═N/O)ccc2ccccc21)

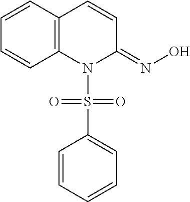 [SMILES: O=S(=O)(c1ccccc1)n1/c(=N/O)ccc2ccccc21]

The benzenesulfonyl is further defined by Benzenesulfonyl chloride is an organosulfur compound. The benzenesulfonyl is further substituted by at least one of one of a halogen, alkyl, alkoxy, nitro, or cyano group. The hydroxylamine is an inorganic compound with the formula NH 2OH. The hydroxylamine is further substituted by at least one of a halogen, alkyl, alkoxy, nitro, or cyano group.

The quinolin is further defined by the bivalent radical C5H3N(CO—)2 of quinolinic acid. The quinolin is further substituted by at least one of a halogen, alkyl, alkoxy, nitro, or cyano group.

The ylidene is further defined by bivalent radical derived from a saturated hydrocarbon by removal of two hydrogen atoms from the same carbon atom or the removal of the oxygen atom of an aldehyde. The ylidene is further substituted by at least one of a halogen, alkyl, alkoxy, nitro, or cyano group.

An advantage of the present invention is to provide innovative organic compound (oxime) used to combat chemicals causing poisoning of a nervous system of a human by inhibiting an enzyme in the nervous system leading to an accumulation of acetylcholine, a neurotransmitter, and overstimulation of the nervous system, leading to symptoms such as muscle twitching, convulsions, and respiratory failure

Another advantage of the present invention is to provide the innovative organic compound (oxime) used in multi-level and multi-modal data (i.e. data ranging from the chemical level, pathways, and systems levels) through state-of-the-art AI-based solutions and provides a valuable opportunity to further understand the molecular mechanisms of CWA exposure in general, as well as patient phenotyping, assessment of patient trajectories as well as the clinical response to interventions at the onset of CWA exposure.

Still another advantage of the present invention is to provide the innovative organic compound (oxime) providing a fast, available and cost-effective platform for treatment design.

Still another advantage of the present invention is to provide the

innovative organic compound (oxime) against nervous agents (organophosphores), which has been tested in-silico for efficacy and safety and is now ready for in-vitro assays.

Still another advantage of the present invention is to provide the innovative organic compound (oxime) that will empower caregivers in the use of novel management tools and combination therapies enabling the adoption of more effective management and personalized care by taking a systems-level approach to fine-tuning treatment and vital support.

The objects and advantages of the present invention will be more readily apparent from inspection of the following specification, taken in connection with the accompanying drawing, wherein like numerals refer to like parts throughout and in which an embodiment of the present invention is described and illustrated.

The exact manner in which the foregoing and other objects and advantages of the invention are achieved in practice will become more clearly apparent when reference is made to the following detailed description of the preferred embodiments of the invention described in detail in the following specification and shown in the accompanying drawings, where in like reference numbers indicate corresponding parts throughout.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 shows a table presenting the affinity of all tested oximes against different AChE inhibited with different organophosphates;

FIG. 2 illustrates in-silico results NE)-N-[1-(benzenesulfonyl) quinolin-2-ylidene]hydroxylamine binding to AChE inhibited with SARIN;

FIG. 3 illustrates in-silico results E)-N-(2,4-dinitrophenoxy)-1-(4-fluorophenyl)methanimine binding to AChE inhibited with VX/Novichok;

FIG. 4 illustrates in-silico results (E)-N-(2,4-dinitrophenoxy)-1-(4-fluorophenyl)methanimine binding to AChE inhibited with SARIN;

FIG. 5 illustrates in-silico results (E)-N-(2,4-dinitrophenoxy)-1-(4-fluorophenyl)methanimine binding to AChE inhibited with VX/Novichok;

FIG. 6 illustrates Retrosynthesis & Synthesis Process of (NE)-N-[1-(benzenesulfonyl)quinolin-2-ylidene]hydroxylamine; and

FIG. 7 illustrates Retrosynthesis & Synthesis Process of (E)-N-(2,4-dinitrophenoxy)-1-(4-fluorophenyl)methanimine.

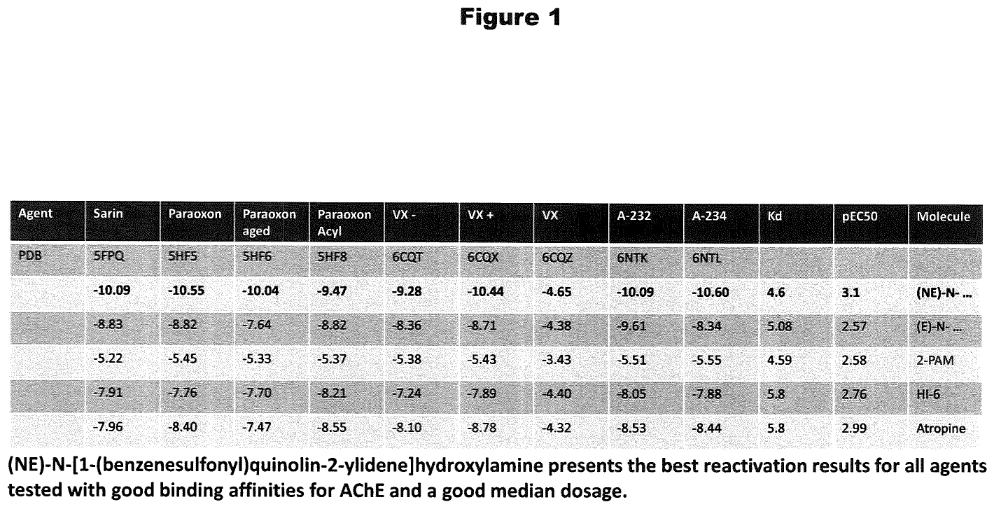

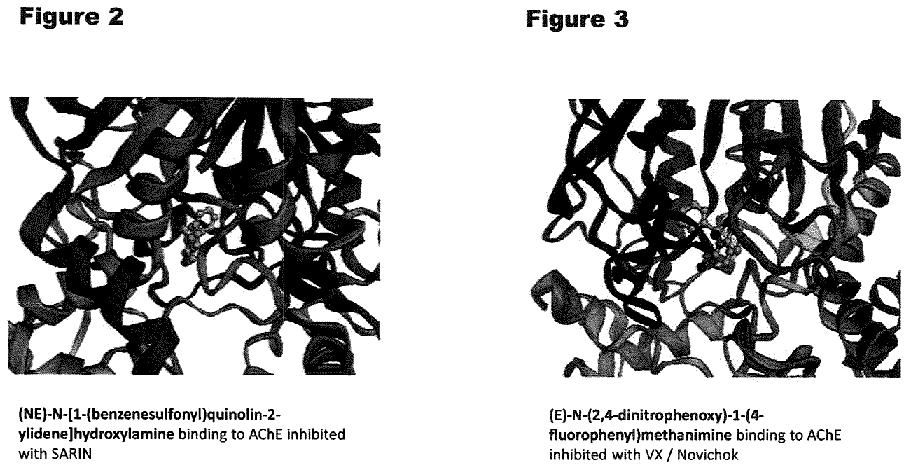

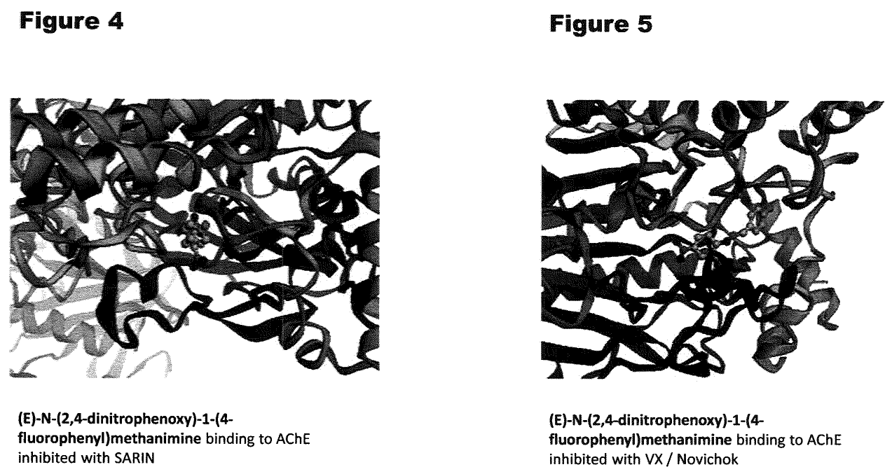

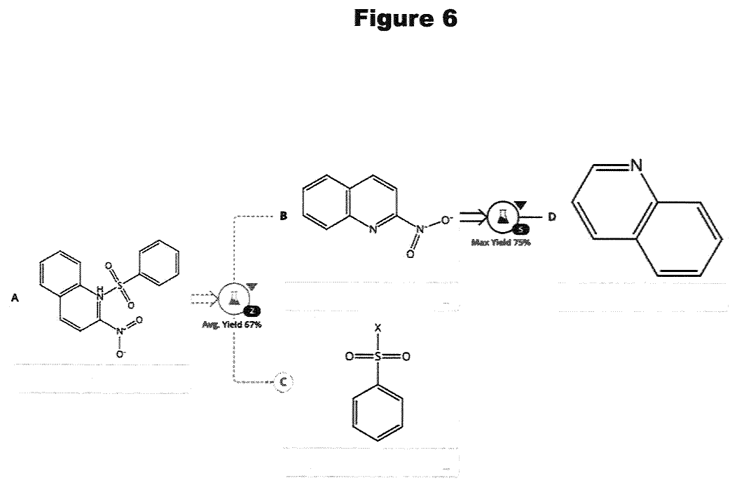

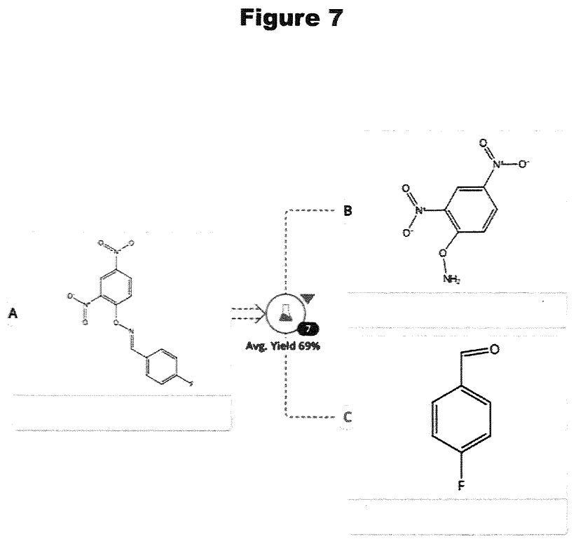

### DETAILED DESCRIPTION OF THE INVENTION

Referring to the Figures, it will be apparent that multiple embodiments of this disclosure may be practiced without some or all of these specific details. In other instances, well-known process operations have not been described in detail in order not to unnecessarily obscure the present embodiments. The following description of embodiments includes references to the accompanying drawing.

The drawing shows illustrations in accordance with example embodiments. These example embodiments, which are also referred to herein as “examples,” are described in enough detail to enable those skilled in the art to practice the present subject matter. The embodiments can be combined, other embodiments can be utilized, or structural, logical and operational changes can be made without departing from the scope of what is claimed. The following detailed description is, therefore, not to be taken in a limiting sense, and the scope is defined by the appended claims and their equivalents.

Alluding to the above, for purposes of this patent document, the terms “or” and “and” shall mean “and/or” unless stated otherwise or clearly intended otherwise by the context of their use. The term “a” shall mean “one or more” unless stated otherwise or where the use of “one or more” is clearly inappropriate. The terms “comprise,” “comprising,” “include,” and “including” are interchangeable and not intended to be limiting. For example, the term “including” shall be interpreted to mean “including, but not limited to.”

An organic compound (oxime) used to combat chemicals causing poisoning of a nervous system of a human by inhibiting an enzyme in the nervous system leading to an accumulation of acetylcholine, a neurotransmitter, and overstimulation of the nervous system, leading to symptoms such as muscle twitching, convulsions, and respiratory failure, said organic compound comprising: (NE)-N-[1-(benzenesulfonyl)quinolin-2-ylidene]hydroxylamine (O═S)(═O)(c1ccccc1)n1/c(═N/O)ccc2ccccc21)

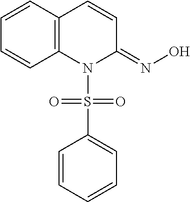 [SMILES: O=S(=O)(c1ccccc1)n1/c(=N/O)ccc2ccccc21]

The benzenesulfonyl is further defined by Benzenesulfonyl chloride is an organosulfur compound. The benzenesulfonyl is further substituted by at least one of one of a halogen, alkyl, alkoxy, nitro, or cyano group. The hydroxylamine is an inorganic compound with the formula NH 2OH. The hydroxylamine is further substituted by at least one of a halogen, alkyl, alkoxy, nitro, or cyano group.

The quinolin is further defined by the bivalent radical C5H3N(CO—)2 of quinolinic acid. The quinolin is further substituted by at least one of a halogen, alkyl, alkoxy, nitro, or cyano group. The ylidene is further defined by bivalent radical derived from a saturated hydrocarbon by removal of two hydrogen atoms from the same carbon atom or the removal of the oxygen atom of an aldehyde. The ylidene is further substituted by at least one of a halogen, alkyl, alkoxy, nitro, or cyano group.

By the way of background and alluding to the above, organophosphates are chemicals that can cause poisoning by inhibiting an enzyme called acetylcholinesterase (AChE) in the nervous system. This leads to an accumulation of acetylcholine, a neurotransmitter, and overstimulation of the nervous system, leading to symptoms such as muscle twitching, convulsions, and respiratory failure.

Oximes in general (and the ones proposed here) work as an antidote for organophosphate poisoning by reactivating the inhibited acetylcholinesterase, thereby reducing the accumulation of acetylcholine and restoring normal nervous system function.

When an organophosphate molecule binds to acetylcholinesterase, it inactivates the enzyme and stops it from breaking down acetylcholine. Oximes are drugs that can help to reverse this inactivation by acting as a “bridge” between the acetylcholinesterase and the organophosphate molecule.

The oxime molecule attaches itself to both the acetylcholinesterase and the organophosphate molecule, helping to break the bond between them. This allows the acetylcholinesterase to regain its normal shape and function, so it can once again break down acetylcholine. This helps to reduce the amount of acetylcholine in the nervous system and relieve the symptoms of organophosphate poisoning.

In essence, the oxime acts as a sort of “intermediary” that helps to restore normal acetylcholinesterase function, thereby reducing the toxic effects of the organophosphate molecule.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. An organic compound (oxime) used to combat chemicals causing poisoning of a nervous system of a human by inhibiting an enzyme in the nervous system leading to an accumulation of acetylcholine, a neurotransmitter, and overstimulation of the nervous system, leading to symptoms such as muscle twitching, convulsions, and respiratory failure, said organic compound comprising:
(NE)-N-[1-(benzenesulfonyl)quinolin-2-ylidene]hydroxylamine (O═S(═O)(c1ccccc1)n1/c(⊚N/O)ccc2ccccc21)

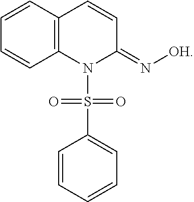 [SMILES: O=S(=O)(c1ccccc1)n1/c(=N/O)ccc2ccccc21]

2. The organic compound (oxime) as set forth in claim 1, wherein said benzenesulfonyl is further defined by Benzenesulfonyl chloride is an organosulfur compound.

3. The organic compound (oxime) as set forth in claim 2, wherein said benzenesulfonyl is further substituted by at least one of one of a halogen, alkyl, alkoxy, nitro, or cyano group.

4. The organic compound (oxime) as set forth in claim 1, wherein said hydroxylamine is an inorganic compound with the formula NH 2OH.

5. The organic compound (oxime) as set forth in claim 4, wherein said hydroxylamine is further substituted by at least one of a halogen, alkyl, alkoxy, nitro, or cyano group.

6. The organic compound (oxime) as set forth in claim 1, wherein said quinolin is further defined by the bivalent radical C5H3N(CO—)2 of quinolinic acid.

7. The organic compound (oxime) as set forth in claim 6, wherein said quinolin is further substituted by at least one of a halogen, alkyl, alkoxy, nitro, or cyano group.

8. The organic compound (oxime) as set forth in claim 1, wherein said ylidene is further defined by bivalent radical derived from a saturated hydrocarbon by removal of two hydrogen atoms from the same carbon atom or the removal of the oxygen atom of an aldehyde.

9. The organic compound (oxime) as set forth in claim 8, wherein said ylidene is further substituted by at least one of a halogen, alkyl, alkoxy, nitro, or cyano group.

10. An organic compound (oxime) used to combat chemicals causing poisoning of a nervous system of a human by inhibiting an enzyme in the nervous system leading to an accumulation of acetylcholine, a neurotransmitter, and overstimulation of the nervous system, leading to symptoms such as muscle twitching, convulsions, and respiratory failure, said organic compound comprising:
(NE)-N-[1-(benzenesulfonyl)quinolin-2-ylidene]hydroxylamine (O═S(═O)(c1ccccc1)n1/c(═N/O)ccc2ccccc21) 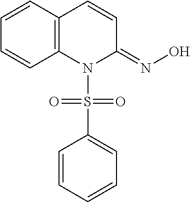 [SMILES: O=S(=O)(c1ccccc1)n1/c(=N/O)ccc2ccccc21]
wherein said benzenesulfonyl is further defined by Benzenesulfonyl chloride is an organosulfur compound, said benzenesulfonyl is further substituted by at least one of one of a halogen, alkyl, alkoxy, nitro, or cyano group;
wherein said hydroxylamine is an inorganic compound with the formula NH 2OH, said hydroxylamine is further substituted by at least one of a halogen, alkyl, alkoxy, nitro, or cyano group;
wherein said quinolin is further defined by the bivalent radical C5H3N(CO—)2 of quinolinic acid, said quinolin is further substituted by at least one of a halogen, alkyl, alkoxy, nitro, or cyano group; and
wherein said ylidene is further defined by bivalent radical derived from a saturated hydrocarbon by removal of two hydrogen atoms from the same carbon atom or the removal of the oxygen atom of an aldehyde, said ylidene is further substituted by at least one of a halogen, alkyl, alkoxy, nitro, or cyano group.
